(19) United States
(12) Patent Application Publication
(10) Pub. No.: US 2007/0023550 A1
(43) Pub. Date: Feb. 1, 2007

(54) Hospital waste treatment with improved disinfectant liquid production

(76) Inventors: Felix F. Jakobi, Long Beach, CA (US); James F. Bennett, La Mirada, CA (US)

Correspondence Address: AVERILL & VARN, 8244 PAINTER AVE., WHITTIER, CA, 90602, US

(21) Appl. No.: 11/212,009

(22) Filed: Aug. 25, 2005

Related U.S. Application Data
(63) Continuation-in-part of application No. 11/190,343, filed on Jul. 26, 2005.

(51) Int. Cl. B02C 19/00; A61L 2/18

(52) U.S. Cl. 241/21; 422/300; 422/62; 422/28; 422/37; 422/3

(57) ABSTRACT

An infectious waste treatment system uses a chlorine dioxide based liquid disinfectant generated from combining precursors comprising sodium chlorite, acid, and bleach. The waste is ground into small pieces and soaked in the liquid disinfectant. The chlorine dioxide is generated by combining dilute aqueous precursors with a flow of the liquid disinfectant into a circulation pump. A preferred set of precursors comprises an approximately 25 percent aqueous sodium chlorite solution, an approximately 12 percent to approximately 50 percent citric acid solution, and an approximately 12 percent industrial bleach (sodium hypochlorite) solution such as Clorox® bleach. A continuous gas monitoring system measures the concentration of chlorine dioxide in the liquid disinfectant and commands the chlorine dioxide generator to generate chlorine dioxide when necessary.

The present application is a Continuation In Part of U.S. application Ser. No. 11/190,343, filed Jul. 26, 2005, for “INFECTIOUS WASTE TREATMENT” which application is incorporated herein by reference.

### BACKGROUND OF THE INVENTION

The present invention relates to a device and method for treatment of waste and in particular to the generation and use of a liquid disinfectant for treating infectious waste.

In the normal course of operation, hospitals generate a variety of waste which is not suitable for normal disposal. While some or most hospital waste may be harmless, it is difficult to distinguish such harmless waste from infectious waste. As a result, all of the waste from a hospital must be treated as if it may be harmful. Also, sensitivity to the handling of hospital waste has been raised as a result of AIDS and other health issues. Recently, the bird flu spread rapidly and initially was not well understood. As world travel has increased, so has the ability of infections, like the bird flu, to spread rapidly, and the need to contain outbreaks is greater than ever before. For all of these reasons, there is a need to deal properly with hospital waste.

Common methods of treating hospital waste include systems having a steam autoclave or an ethylene oxide autoclave. U.S. Pat. No. 6,726,136 for “Waste treatment plant,” describes a system including an autoclave. Other systems include incinerators. Unfortunately, incinerators may be difficult to construct and operate, and may create environmental issues. Autoclaves may also be expensive and difficult to operate. Systems including autoclaves may also require additional steps to complete disinfecting waste.

U.S. Pat. Nos. 5,424,925 and 5,656,248 for “Multi-stage infectious waste treatment system,” both assigned to the assignee of the present application, describe waste treatment systems which grind waste into small particle size, and then soak the waste in a volatile liquid disinfectant. The '248 patent teaches the advantages of using aqueous chlorine dioxide as a liquid disinfectant. The '248 patent further teaches generating chlorine dioxide from a combination of sodium chlorite and a weak organic acid. While there are several advantages in using aqueous chlorine dioxide, the generation of chlorine dioxide from sodium chlorite and a weak organic acid results in a delay in the introduction of chlorine dioxide when a low chlorine dioxide concentration is measured, and in a delay in stopping the production of chlorine dioxide when the desired concentration is reached. Because the infectious waste treatment equipment can only be operated within a range of disinfectant concentrations, periods of non-operation result. The '925 and the '248 patents are herein incorporated by reference.

Other hospital wastes that have been subject to special treatment are Trace Chemo and Suction Canisters. Trace Chemo is trace material left in used waste containers, tubes, needles etc. after use. Trace Chemo medical waste is considered empty waste because the Chemo that was once inside of the waste containers, tubes, needles etc. is no longer inside. Presently, Trace Chemo is treated by burning.

Suction Canisters are filled with fluids and contents from the operating room. Because the contents inside the canister are fluids or gelled fluid, it is difficult for them to reach disinfecting temperatures using known treatment methods. Presently, the only method commonly utilized to treat Trace Chemo and Suction Canisters is incineration.

### BRIEF SUMMARY OF THE INVENTION

The present invention addresses the above and other needs by providing an infectious waste treatment system which uses a chlorine dioxide disinfectant generated by combining precursors comprising sodium chlorite, acid, and bleach. The waste is ground into small pieces and soaked in the chlorine dioxide. The chlorine dioxide is generated by combining dilute aqueous precursors metered by fixed flow restrictors. An efficient set of precursors comprises an approximately 25 percent aqueous sodium chlorite solution, an approximately 12 percent to approximately 50 percent citric acid solution, and approximately a 12 percent industrial bleach (i.e., sodium hypochlorite) solution such as sold under the trademark Clorox®. A continuous gas monitoring system measures the concentration of chlorine dioxide in the system and commands the chlorine dioxide generator to generate chlorine dioxide when necessary. When a low liquid disinfectant level is measured, the chlorine dioxide is generated by combining the precursors with water. When the liquid disinfectant level is acceptable, the chlorine dioxide is generated by combining the precursors with existing liquid disinfectant.

In accordance with one aspect of the invention, there is provided a hospital waste treatment system. The hospital waste treatment system comprises a grinder for receiving unprocessed waste material and grinding the unprocessed waste material to produce ground material, a main solution tank for receiving the ground material from the grinder, and a liquid disinfectant in the main solution tank for disinfecting the ground material. The liquid disinfectant comprises an aqueous chlorine dioxide solution. The aqueous chlorine dioxide is generated from precursors comprising aqueous sodium chlorite, an acid, and a bleach. A chlorine dioxide generator is used for combining the precursors with a flow to generate the aqueous chlorine dioxide to add to the liquid disinfectant. The chlorine dioxide generator includes an eductor for drawing the precursors into a flow and the amount of the individual precursors drawn into the eductor is regulated by fixed flow restrictors.

In accordance with another aspect of the invention, there is provided a hospital waste treatment system. The hospital waste treatment system comprises a grinder for receiving unprocessed waste material and grinding the unprocessed waste material to produce ground material, a main solution tank for receiving the ground material from the grinder, and a liquid disinfectant in the main solution tank for disinfecting the ground material. The liquid disinfectant comprising aqueous chlorine dioxide generated from precursors. The precursors used to generate the aqueous chlorine dioxide comprise aqueous sodium chlorite, citric acid, and aqueous sodium hypochlorite. A chlorine dioxide generator is used for combining the precursors with a flow to generate aqueous chlorine dioxide to add to the liquid disinfectant.

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1 is a waste treatment system including a chlorine dioxide generator according to the present invention.

FIG. 2A is a side view of a main solution tank suitable for use with the waste treatment system.

FIG. 2B is a top view of the main solution tank.

FIG. 3 is a cross-sectional view of the main solution tank taken along line 3-3 of FIG. 2B.

FIG. 4 is a second side view of the waste treatment system (opposite side) showing a gas monitoring system, a main solution tank circulation pump, and a liquid disinfectant generation elements.

FIG. 5 is a detailed view a chemical manifold.

FIG. 6 is a method for treating hospital waste according to the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

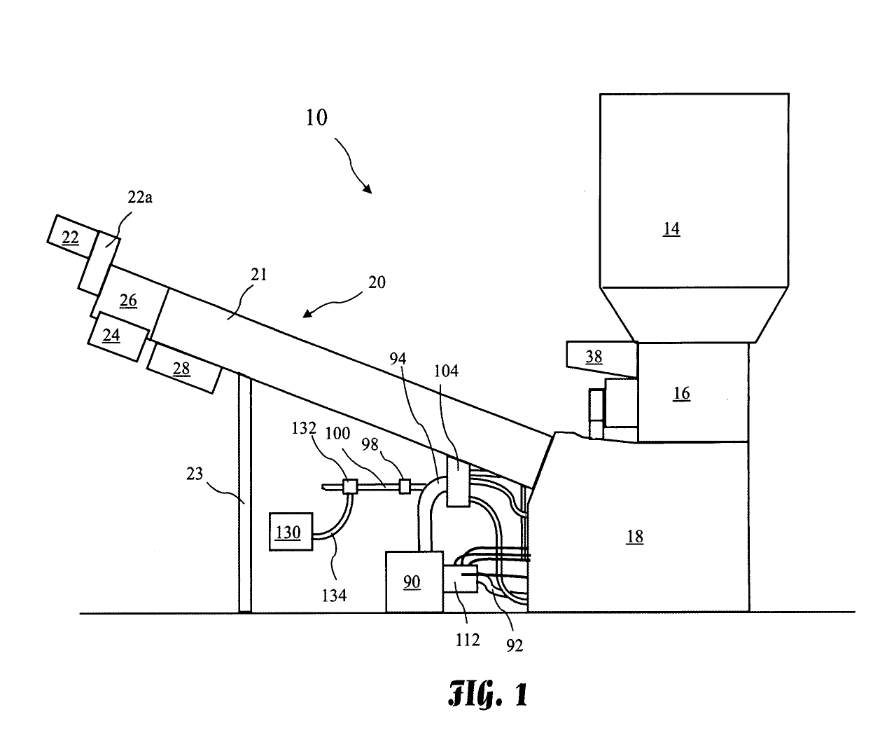

A waste treatment system 10 according to the present invention is shown in FIG. 1. The waste treatment system 10 includes a hopper 14, a grinder 16, a main solution tank 18, and an auger 20. Hospital waste is introduced into the hopper 14. The hopper 14 resides above the grinder 16 and feeds the waste into the grinder 16. The grinder 16 grinds the waste, and the ground waste drops into the main solution tank 18 where the ground waste is wetted. The auger 20 lifts the wetted waste from the main solution tank 18 and completes the waste treatment.

Continuing with FIG. 1, a pump 90 receives disinfectant liquid from the main solution tank 18 through a pump inlet line 92, and returns the disinfectant liquid through a pump outlet line 94 through a manifold 104. A drain line 100 is connected to the pump outlet line 94 through a drain valve 98. A neutralizer tank 130 is connected to the drain line 100 at a neutralizer injector 130 for neutralizing the drained disinfectant liquid. The pump 90 is preferably a chopper pump, and is more preferably a high flow rate pump, and most preferably an approximately 200 Gallon Per Minute (GPM) pump. An example of a suitable 200 GPM pump is a model number HE3G6SEC-055 chopper pump manufactured by Vaughn Company in Montesano, Wash. In some cases, separate pumps may be used to recycled the disinfectant liquid and to spray the disinfectant liquid onto the waste material. When two pumps are used, the pumps are preferably approximately 90 Gallon Per Minute (GPM) pumps.

A continuous gas monitoring system 38 monitors the liquid disinfectant level in the main solution tank 18 and composition (i.e., strength) of the liquid disinfectant, and controls the generation of liquid disinfectant (see FIG. 8). For example, chemicals may be introduced into a flow into the pump 90 at a chemical manifold 112 to generate liquid disinfectant. An example of a continuous gas monitoring system 38 is the system described in U.S. Pat. No. 5,269,832 for “Method and Apparatus for Continuously Measuring the Concentration of Chemicals in Solution.” The '832 patent is herein incorporated by reference.

The auger 20 is preferably a shaftless auger residing in an auger housing 21 supported by an auger strut 23 and is powered by an auger motor 22 which is preferably connected to the auger 20 through a gearbox 22a. The auger 20 further includes a fluid trap 28 where the liquid disinfectant used to wet the ground waste is trapped and recirculated back into the main tank. A rotatable section 26 of the auger housing 21 may be rotationally positioned relative to the auger housing 21 at various rotations to adjust the position of a chute 24. If the chute 24 is pointed down, the back pressure on the flow of the ground waste is minimized, and the amount of liquid disinfectant removed by the fluid trap 28 is minimized. As the chute 24 is rotated away from a pointed down position, the back pressure on the flow of the ground waste is increased, and the amount of liquid disinfectant removed by the fluid trap 28 is increased. If the chute 24 is rotated to an upward position, the back pressure on the flow of the ground waste is maximized, and the amount of liquid disinfectant removed by the fluid trap 28 is maximized.

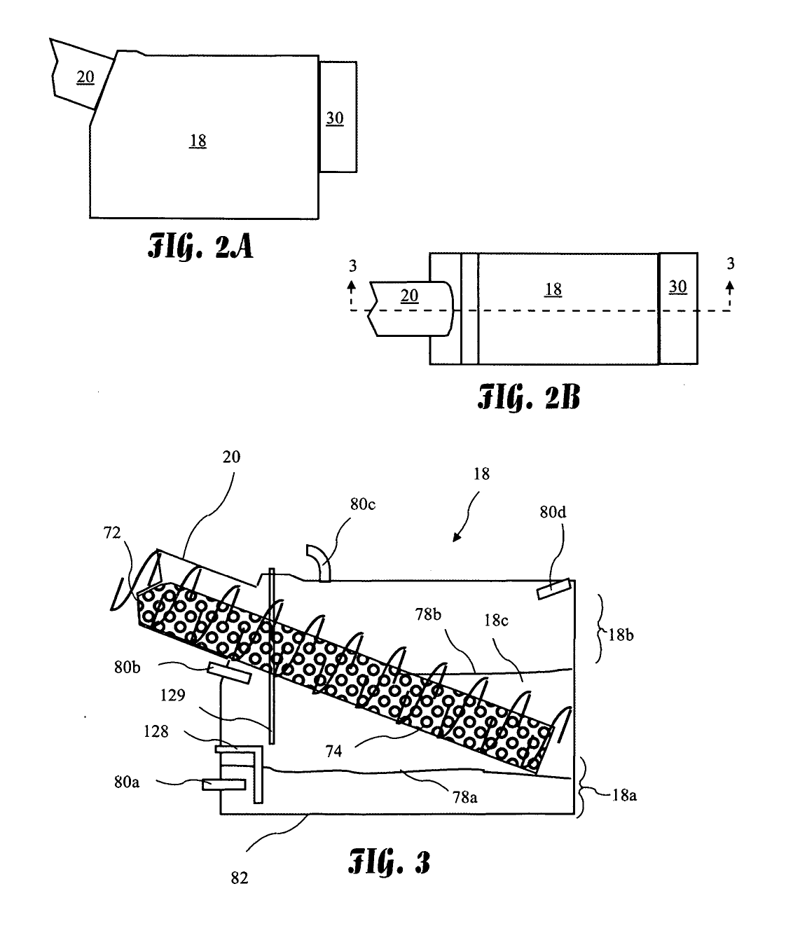

A side view of the main solution tank 18 suitable for use with the waste treatment system 10 is shown in FIG. 2A, a top view of the main solution tank 18 is shown in FIG. 2B. A cross-sectional view of the main solution tank 18 taken along line 3-3 of FIG. 2B is shown in FIG. 3. An auger screw 72 extends though the main solution tank 18 and is cupped by an auger floor 74 which is preferably an auger screen extending under approximately half of the circumference of the auger screw 72. The liquid disinfectant resides in a lower portion 18a of the main solution tank 18 with a static fluid level 78a. Additionally, while the waste treatment system 10 is in operation, the liquid disinfectant resides at a dynamic level 78b above the auger floor 74 in a wetting portion 18c of the main solution tank 18. The dynamic liquid level 78b is maintained in equilibrium by the cooperation of pumping the liquid disinfectant into an upper portion 18b of the main solution tank 18 and the liquid disinfectant draining through the auger floor 74 into the lower portion 18a of the main solution tank.

Continuing with FIG. 3, a first nozzle 80a provides a flow of the liquid disinfectant into the lower portion 18a of the main solution tank to provide circulation of the liquid disinfectant, a second nozzle 80b provides a flow of the liquid disinfectant into the upper portion 18b of the auger end of the main solution tank 18, a third nozzle 80c provides a flow of the liquid disinfectant into the upper portion 18b of the main solution tank 18 near the auger end of the main solution tank 18 (i.e., where the auger 20 enters the main solution tank 18), and a fourth nozzle 80d is positioned opposite the auger end of the main solution tank 18 and provides a flow of the liquid disinfectant directed towards the auger screw 72.

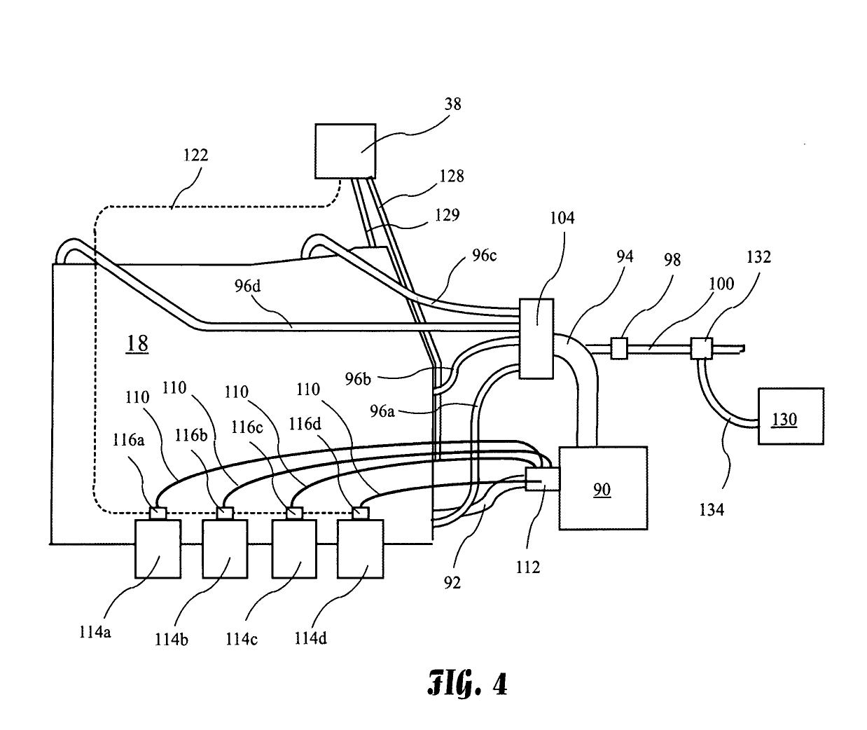

A bubble tank assembly 128 is partially submerged in the disinfectant liquid below the static fluid level 78a and to preferably within approximately one half inch of the bottom of the main solution tank 18, and is further described in FIG. 4. The bubble tank assembly 128 measures the liquid disinfectant depth. A gas sample tube 129 resides in the main solution tank 18 and has a lower end above the static fluid level 78a, and preferably between approximately six inches and approximately eight inches above the static fluid level 78a.

A second side view of the main solution tank 18 (an opposite side view from FIGS. 1 or 3) showing the continuous gas monitoring system 38, the pump 90, and liquid disinfectant generator elements are shown in FIG. 4. The pump 90 draws the liquid disinfectant from the lower portion 18a of the main solution tank 18 through the inlet line 92 and returns the liquid disinfectant to the nozzles 80a, 80b, 80c, and 80e (see FIG. 3) through nozzles lines 96a, 96b, 96c, and 96e respectively connected to the circulation pump 90 by the outlet line 94 through the manifold 104. The drain valve 98 is also connected to the outlet line 94, and a drain line 100 is connected to the drain valve 98 to allow convenient draining of the main solution tank 18. A neutralizer tank 130 is connected to a neutralizer nozzle 132 in the drain line 100 by a neutralizer line 134. The neutralizer neutralizes the disinfectant liquid, and is preferably sodium sulfite.

Continuing with FIG. 4, the continuous gas monitoring system 38 measures the gas concentration using the gas sample tube 129 (also see FIG. 3). The continuous gas monitoring system 38 provides control signals over a control cable 122 to valves or pumps 116a, 116b, 116c, and 116d to control a flow of liquid disinfectant precursors from chemical tanks 114a, 114b, 114c, and 114d to a second manifold 112. The valves or pumps 116a, 116b, 116c, and 116d are preferably pump, and more preferably four peristaltic pumps with a preferable flow rate between approximately 50 Gallons Per Day (GPD) and approximately 150 GPD, and more preferably approximately 95 GPD. An example of an appropriate peristaltic pump is a Model No. A1N30V-7T pump made by Blue-White Industries in Huntington Beach, Calif. The Model No. A1N30V-7T pumps are variable rate pumps, and are preferably set to maximum rate if used.

The liquid disinfectant precursors preferably comprise an approximately 12 percent industrial clorox bleach (i.e., sodium hypochlorite), an approximately 12 percent to approximately 50 percent citric acid solution, an approximately 25 percent sodium chlorite solution as precursors for chlorine dioxide, and an anti-form agent. The chemical manifold 112 is in serial fluid communication between the main solution tank and the pump inlet, thus introducing the precursors into a flow of the liquid disinfectant into the pump 90.

The continuous gas monitoring system 38 includes a continuous gas monitoring device which uses a diaphragm pump to provide the gas flow received through the gas sample tube 129 to a sensor. The sensor's electrical output is sent through a sensor circuit board to a digital panel meter which processes the sensor output and produces a digital readout in Parts Per Million (PPM) of the chemical levels in the liquid disinfectant. The continuous gas monitoring system 38 compares the measured gas level to the preset alarm levels and activates alarm indicators when gas levels exceed user set levels. If low gas levels are detected, a signal is sent to the liquid disinfectant generator to generate additional chlorine dioxide. If the liquid disinfectant is low, water is added to the systems. The continuous gas monitoring system 38 further includes data logging for recording data including chemical levels, fluid level, maintaining level, and kill ratio.

The static liquid level 78a (see FIG. 3) of the liquid disinfectant in the main solution tank 18 is measured using the bubble tank assembly 128 (see FIG. 3). The bubble tank assembly 128 comprises a six-inch cylinder sealed at the top with a one half inch tube protruding through the top of the seal and extending one half-inch past the bottom of the cylinder. A second one half-inch tube extends just through the seal into the top of the cylinder. The bubble tank assembly 128 is submerged in the liquid disinfectant in the main solution tank 18 to a depth wherein the longer tube is approximately one half-inch from the bottom of the main solution tank 18. Low-volume air is injected through the longer tube and the resulting pressure inside the cylinder is measured and converted to a measurement of depth of the liquid disinfectant in the main solution tank 18.

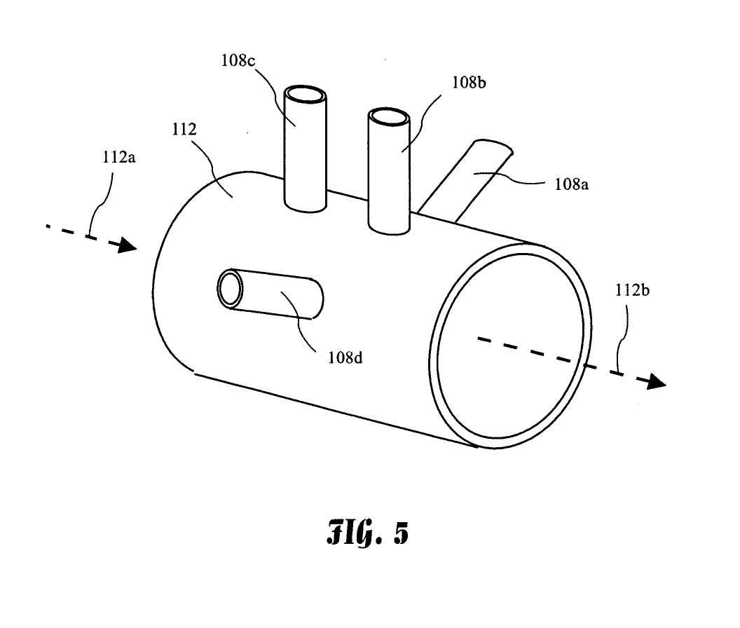

A detailed view of the chemical manifold 112 is shown in FIG. 5. A first flow of liquid disinfectant 112a from the main solution tank 18 enters the manifold 112, and a second flow of liquid disinfectant 112b leaves the manifold 112 to enter the pump 90 (see FIG. 4). Chemical nozzles 108a, 108b, 108c, and 108d provide the liquid disinfectant precursors to the manifold 112. The liquid disinfectant precursors are thus introduced to a liquid disinfectant flow just prior to the flow entering the pump 90, where the liquid disinfectant precursors are mixed within the pump 90.

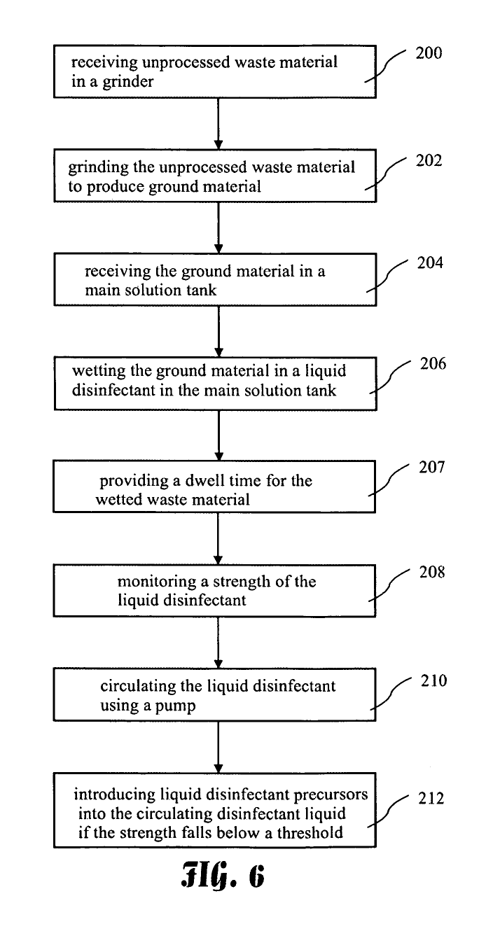

A method for treating hospital waste according to the present invention is described in FIG. 6. The method includes receiving unprocessed waste material in a grinder at step 200, grinding the unprocessed waste material to produce ground material at step 202, receiving the ground material in a main solution tank at step 204, wetting the ground material in a liquid disinfectant in the main solution tank at step 206, monitoring a strength of the liquid disinfectant at step 208, circulating the liquid disinfectant using a pump at step 210, and introducing liquid disinfectant precursors into the circulating disinfectant liquid if the strength falls below a threshold at step 212. The step of introducing liquid disinfectant precursors preferably comprises introducing sodium chlorite, acid, and bleach into the circulating disinfectant liquid, and more preferably comprises introducing an approximately 25 percent aqueous sodium chlorite, a between approximately 12 percent and approximately 50 percent citric acid solution, and an approximately twelve percent aqueous sodium hypochlorite. The step of introducing liquid disinfectant precursors also preferably comprises introducing liquid disinfectant precursors into the circulating disinfectant liquid proximal to the entry of the liquid disinfectant into the pump. The method may further include providing a dwell time for the wetted waste material at step 207, and preferably includes providing a dwell time by carrying the wetted material from the main solution tank on an auger.

The method of the present invention may further be exercised to treat Trace Chemo and Suction Canister hospital waste. Trace Chemo is trace material left in used waste containers, tubes, needles etc. after use. Trace Chemo medical waste is considered empty waste because the chemo that was once inside of the waste containers, tubes, needles etc. is no longer inside. Because Trace Chemo waste is considered empty waste, it simply needs to be ground or burned. Because the present invention grinds the waste that is introduced to it, Trace Chemo can be processed using the method described in FIG. 6.

Suction Canisters are filled with fluids and contents from the operating room. Because the contents inside the canister are fluids or gelled fluid, it is difficult for them to reach disinfecting temperatures. Using the method of the present invention, the Suction Canister is broken open and all of the contents inside become in direct contact with the liquid disinfectant which disinfects the contents.

While the invention herein disclosed has been described by means of specific embodiments and applications thereof, numerous modifications and variations could be made thereto by those skilled in the art without departing from the scope of the invention set forth in the claims.

I claim:

1. Apparatus for hospital waste treatment, the apparatus comprising:
a grinder for receiving unprocessed waste material and grinding the unprocessed waste material to produce ground material;
a main solution tank for receiving the ground material from the grinder and wetting the ground material with a liquid disinfectant;
a pump for circulating a flow of the liquid disinfectant; and
a chemical manifold in fluid communication with the flow of the liquid, the chemical manifold receiving liquid disinfectant precursors to mix with the flow of the liquid disinfectant.

2. The apparatus of claim 1, wherein the precursors comprise sodium chlorite, acid, and bleach.

3. The apparatus of claim 2, wherein the acid is citric acid.

4. The apparatus of claim 2, wherein the bleach is sodium hypochlorite.

5. The apparatus of claim 2, wherein:
the sodium chlorite comprises dilute aqueous sodium chlorite;
the acid comprises a dilute acid; and
the bleach comprises a dilute bleach.

6. The apparatus of claim 2, wherein:
the sodium chlorite comprises an approximately 25 percent aqueous sodium chlorite;
the acid comprises between approximately 12 percent and approximately 50 percent citric acid solution; and
the bleach comprises approximately twelve percent aqueous sodium hypochlorite.

7. The apparatus of claim 1, further including a continuous gas monitoring system for measuring the strength of the liquid disinfectant in the apparatus, wherein the flow of the precursors to the manifold is controlled by the continuous gas monitoring system.

8. The apparatus of claim 1, wherein the chemical manifold further received anti-foam.

9. A hospital waste treatment system comprising:
a grinder for receiving unprocessed waste material and configured to grind the unprocessed waste material to produce ground material;
a main solution tank for receiving the ground material from the grinder and containing a volume of liquid disinfectant in a path of the ground material;
a pump with an inlet in fluid communication with the main solution tank and with an outlet in fluid communication with the main solution tank;
a chemical manifold in serial fluid communication between the main solution tank and the pump inlet; and
precursor tanks in fluid communication with the chemical manifold, the precursor tanks comprising:
an aqueous sodium chlorite tank;
a citric acid tank; and
an aqueous sodium hypochlorite tank.

10. The apparatus of claim 9, wherein:
the sodium chlorite comprises an approximately 25 percent aqueous sodium chlorite;
the acid comprises between approximately 12 percent and approximately 50 percent citric acid solution; and
the bleach comprises approximately twelve percent aqueous sodium hypochlorite.

11. A method for treating hospital waste, the method comprising:
receiving unprocessed waste material in a grinder;
grinding the unprocessed waste material to produce ground material;
receiving the ground material in a main solution tank;
wetting the ground material in a liquid disinfectant in the main solution tank;
monitoring a strength of the liquid disinfectant;
circulating the liquid disinfectant using a pump; and
introducing liquid disinfectant precursors into the circulating disinfectant liquid if the strength falls below a threshold.

12. The method of claim 11, wherein introducing liquid disinfectant precursors comprises introducing sodium chlorite, acid, and bleach into the circulating disinfectant liquid.

13. The method of claim 12, wherein introducing liquid disinfectant precursors comprises introducing an approximately 25 percent aqueous sodium chlorite, a between approximately 12 percent and approximately 50 percent citric acid solution, and an approximately twelve percent aqueous sodium hypochlorite.

14. The method of claim 11, wherein introducing liquid disinfectant precursors into the circulating disinfectant liquid comprises introducing liquid disinfectant precursors into the circulating disinfectant liquid proximal to the entry of the liquid disinfectant into the pump.

15. The method of claim 11, further providing a dwell time for the wetted waste material.

16. The method of claim 11, wherein providing a dwell time for the wetted waste material comprises carrying the wetted material from the main solution tank on an auger.
